(19) United States
(12) Patent Application Publication
(10) Pub. No.: US 2015/0217091 A1
(43) Pub. Date: Aug. 6, 2015

(54) TOOL FOR LOADING A MEDICAL DEVICE ONTO A GUIDEWIRE

(71) Applicant: Calore Medical Ltd., Or Akiva (IL)

(72) Inventors: Giora Kornblau, Binyamina (IL); David Maier Neustadter, Nof Ayalon (IL)

(73) Assignee: Calore Medical Ltd.

(21) Appl. No.: 14/616,027

(22) Filed: Feb. 6, 2015

Related U.S. Application Data
(60) Provisional application No. 61/936,347, filed on Feb. 6, 2014.
(60) Provisional application No. 62/015,968, filed on Jun. 23, 2014.

(51) Int. Cl. A61M 25/09

(52) U.S. Cl. CPC A61M 25/09041; A61B 2017/1205

(57) ABSTRACT

A loading tool that aids in single-handed backloading or sideloading of a medical device having a lumen onto a guidewire is described. The loading tool comprises two continuous sections—a proximal attachment section and a distal loading section. The attachment section has a tubular configuration with a longitudinal slot removed. The attachment section is configured to fit over a distal portion of the medical device. The loading section has a conical shape with a longitudinal slot removed. The loading section is configured to guide the proximal end of a guidewire into the lumen of the medical device onto which the attachment section of the loading tool is mounted.

### CROSS-REFERENCES TO RELATED APPLICATIONS

This application claims priority to U.S. Provisional Application No. 61/936,347 filed Feb. 6, 2014, and U.S. Provisional Application No. 62/015,968, filed Jun. 23, 2014, both of which are incorporated herein by reference in their entirety.

### TECHNICAL FIELD

The present disclosure relates generally to a guidewire loading tool, and more specifically, to a tool that aids in loading a medical device having a lumen onto a guidewire pre-inserted within a patient.

### BACKGROUND

During various medical procedures, there is often a need to load a medical device having a lumen, for example, introducer sheath, catheter, vascular closure device, etc., onto a guidewire, which guides the insertion and advancement of the medical device into a blood vessel or to a treatment site within a patient's body. The lumen of the medical device serves to receive the guidewire. The proximal end of an indwelling guidewire is threaded into the distal opening of the medical device lumen and the medical device is then advanced through the patient's body along the guidewire. Guidewires are generally very flexible and have small cross-sectional diameters, which make it very difficult to backload a medical device having a narrow lumen onto a guidewire.

The difficulty in backloading a medical device onto a guidewire is exacerbated if the physician is required to keep one hand at the puncture site, either to apply compressive pressure to minimize bleeding, or to maintain appropriate tension on the guidewire. It is particularly difficult to single-handedly backload a medical device onto a guidewire if the distal tip of the medical device cannot be held between the thumb and the forefinger of the physician either due to the size of the tip or its sensitive nature. The problem of backloading a medical device onto a guidewire is further worsened if the length of the proximal end of the guidewire extending out of the patient's body is relatively short, e.g., only 20-50 cm. Moreover, backloading of the medical device generally has to be done quickly and efficiently, because most treatment procedures preferably have to be implemented within a short span of time to reduce unnecessary stress on the patient.

Therefore, devices and mechanisms that facilitate loading of medical devices onto guidewires are necessary. In particular, devices and mechanisms designed to assist in single-handed loading of a medical device onto a short guidewire are highly desirable.

### SUMMARY

The present disclosure is directed to a loading device that may allow single-handed backloading or sideloading of a medical device having a lumen (referred to as “medical conduit” hereinafter) onto a guidewire, which then guides the medical conduit to a treatment site. It is to be understood that the term “medical conduit” in this disclosure refers to any medical device having a lumen therein, e.g., introducer sheath, catheter, vascular closure device, etc. It is also to be understood that the term “guidewire” in this disclosure includes the shaft of an arterial anchor or artery locator, or any shaft, channel, wire, or elongated element that is used functionally as a guidewire to direct a medical conduit into a blood vessel or to a treatment site within a patient's body.

It is further to be understood that the terms “distal” and “proximal” are used in this disclosure with respect to a position or direction relative to the user, e.g., a physician, implementing the treatment procedure. “Distal” or “distally” are positions distant from or in a direction away from the user. “Proximal” or “proximally” are positions near or in a direction towards the user.

Consistent with a disclosed embodiment, a device is disclosed for loading a medical conduit onto a guidewire. The device may include an incomplete tubular attachment portion configured to fit over an end portion of the medical conduit. The tubular attachment portion may have a longitudinal section removed, and an incomplete conical loading portion may extend from the tubular attachment portion. The conical loading portion may also have a longitudinal section removed. The conical loading portion of the device may be configured to direct the guidewire into a lumen of the medical conduit. Further, the device may be configured to be detachable from the medical conduit after the guidewire is directed into the medical conduit.

Other embodiments of this disclosure are disclosed in the accompanying drawings, description, and claims. Thus, this summary is exemplary only, and is not to be considered restrictive.

### BRIEF DESCRIPTION THE OF DRAWINGS

The accompanying drawings, which are incorporated in and constitute a part of this specification, illustrate the disclosed embodiments and together with the description, serve to explain the principles of the various aspects of the disclosed embodiments. In the drawings:

FIGS. 1A-1E illustrate exemplary embodiments of a loading device of the present disclosure;

FIG. 2 illustrates various features of an exemplary loading device, in accordance with exemplary embodiments of the present disclosure;

FIG. 3 illustrates an exemplary loading device attached to an exemplary medical conduit, in accordance with exemplary embodiments of the present disclosure;

FIG. 4 illustrates various features of an exemplary loading device, in accordance with exemplary embodiments of the present disclosure; and

FIGS. 5A and 5B illustrate the point and angle of contact between an exemplary loading device and an exemplary medical conduit, in accordance with exemplary embodiments of the present disclosure,

It is to be understood that both the foregoing general description and the following detailed description are exemplary and explanatory only and are not restrictive of the disclosed embodiments, as claimed.

### DESCRIPTION OF EXEMPLARY EMBODIMENTS

Reference will now be made to certain embodiments consistent with the present disclosure, examples of which are illustrated in the accompanying drawings. Wherever possible, the same reference numbers are used throughout the drawings to refer to the same or like parts.

The present disclosure describes a loading device that may be mounted at or near the distal end of a medical conduit. The medical conduit is intended to be loaded onto a guidewire with the aid of the loading device. The disclosed loading device, and the mechanisms for loading a medical conduit onto a guidewire with the aid of the loading device, may be employed for any medical procedure. In one particular example, the loading device may be used to load a vascular closure device onto the shaft of an arterial anchor or artery locator to close or heal perforations, openings, or punctures in an artery.

Exemplary embodiments of a loading device of the present disclosure may comprise two continuous sections—a proximal attachment section and a distal loading section. The attachment section may be configured to mount the loading device at or near the distal end of the medical conduit. In exemplary embodiments, the attachment section may have a tubular configuration with a longitudinal portion removed. The attachment section may be configured to fit over a distal portion of the medical conduit. In some embodiments, the outer diameter of the medical conduit may be approximately equal to the inner diameter of the attachment section so that the loading device may be fitted securely to the medical conduit. The attachment section may be further configured to align the lumen of the medical conduit with the proximal end of the loading section.

The loading section may be configured to guide the proximal end of a guidewire (extending outside a patient's body) into the lumen of the medical conduit onto which the attachment section of the loading device is mounted. In exemplary embodiments, the loading section may have a conical shape, e.g., the shape of a funnel, with a longitudinal portion removed. The distal tip of the loading section, and/or the removed longitudinal portion of the loading section, may serve as the guidewire insertion end and the proximal opening of the attachment section serves as the medical conduit insertion end. A continuous lumen may extend throughout the loading device from the guidewire insertion end to the medical conduit insertion end.

A loading device of the present disclosure may be configured to be partially or completely detachable from the medical conduit once the medical conduit is loaded onto the guidewire.

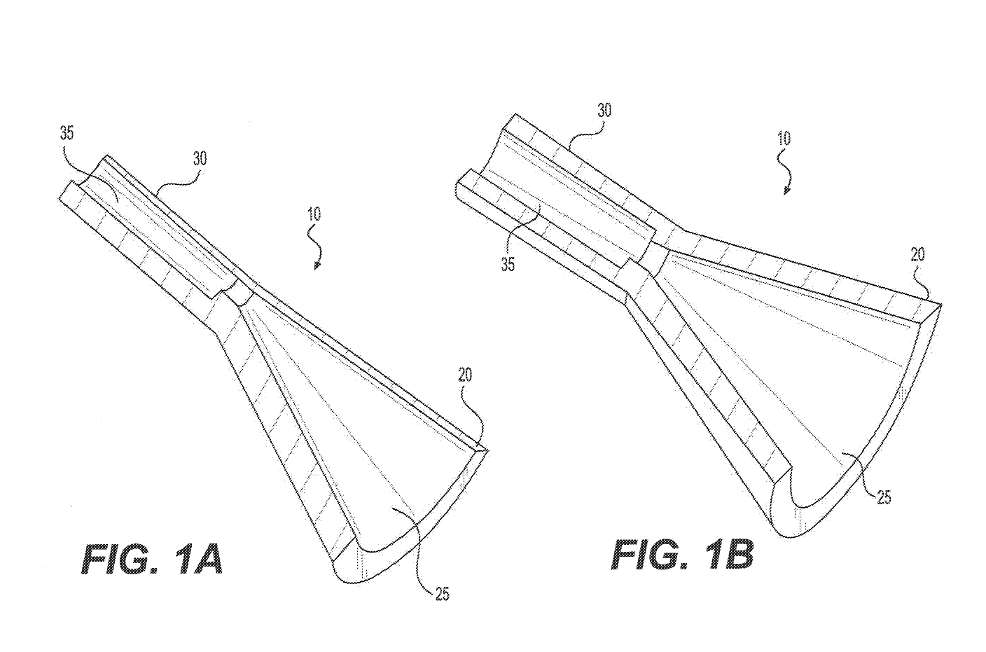

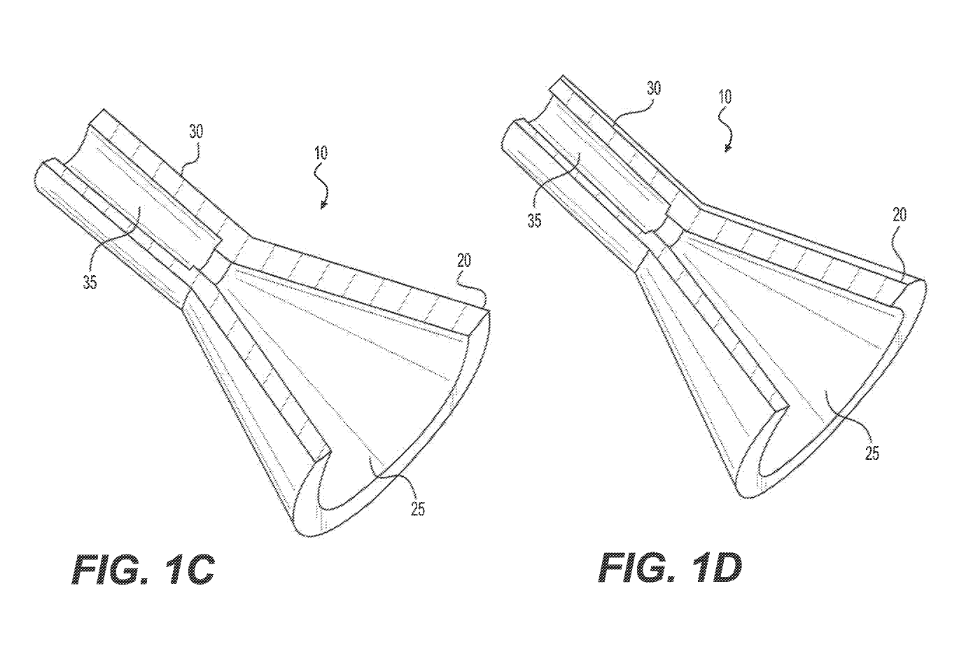

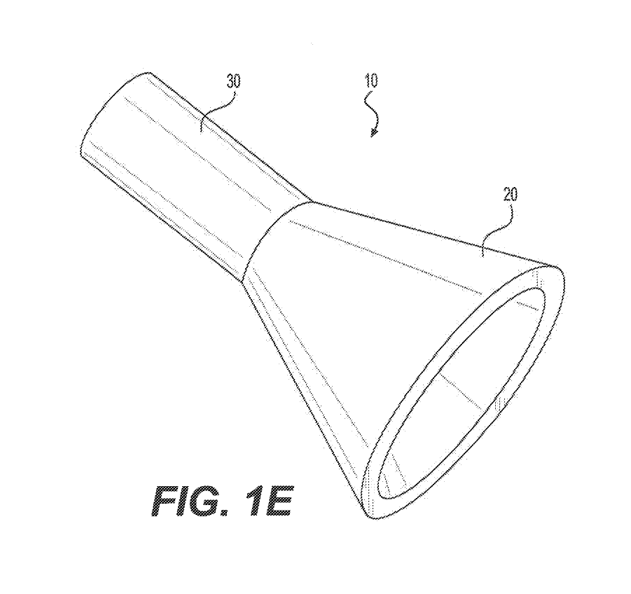

In exemplary embodiments, the loading section may be shaped like a cone or portion of a cone, such as half of a cone, one-third of a cone, or any other fraction of a cone. FIGS. 1A-1E show exemplary embodiments of a loading device 10 of the present disclosure having a tubular attachment section 30 and a cone-shaped loading section 20 that extends from attachment section 30. As shown in FIGS. 1A-1D, a distal longitudinal slot 25 of the cone-shaped loading section 20 may be removed. Distal longitudinal slot 25 may be of different width; for instance, as shown in FIGS. 1A-1D, distal longitudinal slot 25 may constitute half of a cone, one-third of a cone, or any other fraction of cone-shaped loading section 20. Further, as shown in FIGS. 1A-1D, a proximal longitudinal slot 35 of attachment section 30 may also be removed in such a way that distal longitudinal slot 25 and proximal longitudinal slot 35 are aligned. In some embodiments, the same fraction of loading section 20 and attachment section 30 may be removed. For example, if distal longitudinal slot 25 constitutes one-third of loading section 20, then proximal longitudinal slot 35 also constitutes one-third of attachment section 30.

In some exemplary embodiments, distal longitudinal slot 25 may be provided along the side of loading section 20 to allow the guidewire to be inserted into loading device 10 from the side instead of the distalrnost opening of loading section 20. This method of loading the guidewire may be referred to as “side loading.” In the “side loading” approach, the proximal end of the guidewire may be easily inserted or scooped up into loading section 20 and then guided into the distal tip of the medical conduit onto which attachment section 30 is mounted.

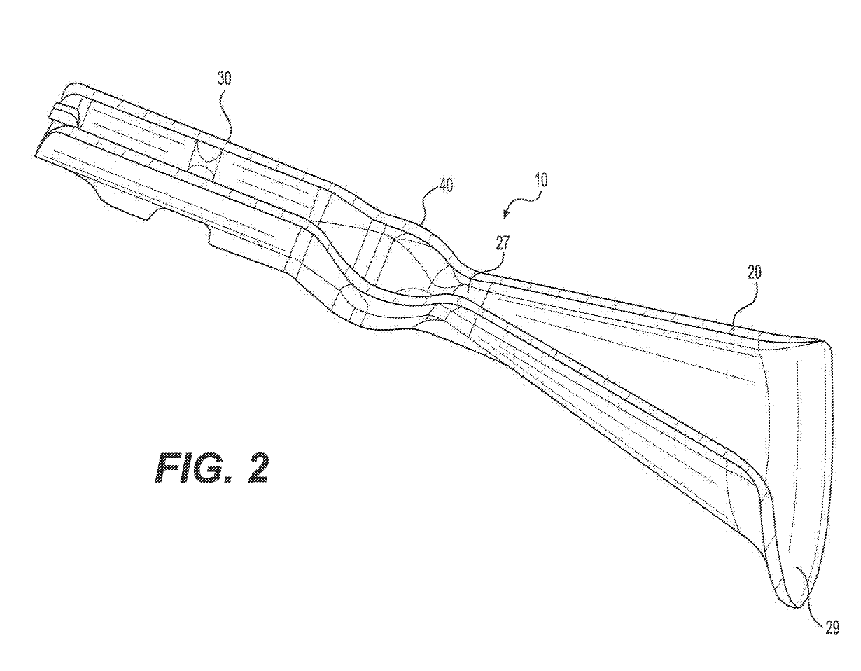

FIG. 2 further illustrates an exemplary embodiment of loading device 10 having a loading section 20 and an attachment section 30. The distal end of attachment section 30 may include an enlarged portion 40, as shown in FIG. 2. Enlarged portion 40 may have a width larger than that of the remaining attachment section 30. Enlarged portion 40 may be provided and configured to accommodate the distal tip of a medical conduit, which may have a diameter/width larger than that of the rest of the medical conduit. For example, if the medical conduit to be loaded is a thermal tissue closure device, then the heating tip of the tissue closure device may be placed in enlarged portion 40. An opening 27 at the proximal end of loading section 20 is aligned with the distal opening of the lumen of the medical conduit (not shown) onto which attachment section 30 is mounted.

In exemplary embodiments, a distal tip 29 of loading section 20 extends further distally at its midpoint than at its sides, as shown in FIG. 2. This may cause the midpoint of distal tip 29 to contact the patient's body first, which may ensure that the forces applied to loading device 10 by contacting patient's body are in the proximal direction causing loading device 10 to pivot/rotate radially outward and detach completely or partially from the medical conduit.

In exemplary embodiments, loading device 10 may be provided on (i.e., attached to) and shipped with a medical conduit to assist with rapid set-up and use by a physician. In some other embodiments, loading device 10 may be supplied separate from the medical conduit, and a physician may connect attachment section 30 of loading device 10 onto the medical conduit prior to use.

In some embodiments, loading device 10 may be removable from the medical conduit following insertion of the guidewire into the lumen of the medical conduit. In such embodiments, loading device 10 may be re-sterilized and reused in multiple treatment procedures. In other embodiments, loading device 10 may be a single-use device that is provided with a single-use medical conduit. In such embodiments, loading device 10 may or may not be removable from the medical device following use.

In exemplary embodiments, attachment section 30 may be configured such that if force is applied in the proximal direction to the distal end of loading section 20, then loading device 10 detaches completely from the medical conduit to which it is attached.

In some embodiments, when the distal end of loading device 10 contacts the patient's body surface, force may be applied to loading device 10 in the proximal direction, which may then cause loading device 10 to detach from the medical conduit.

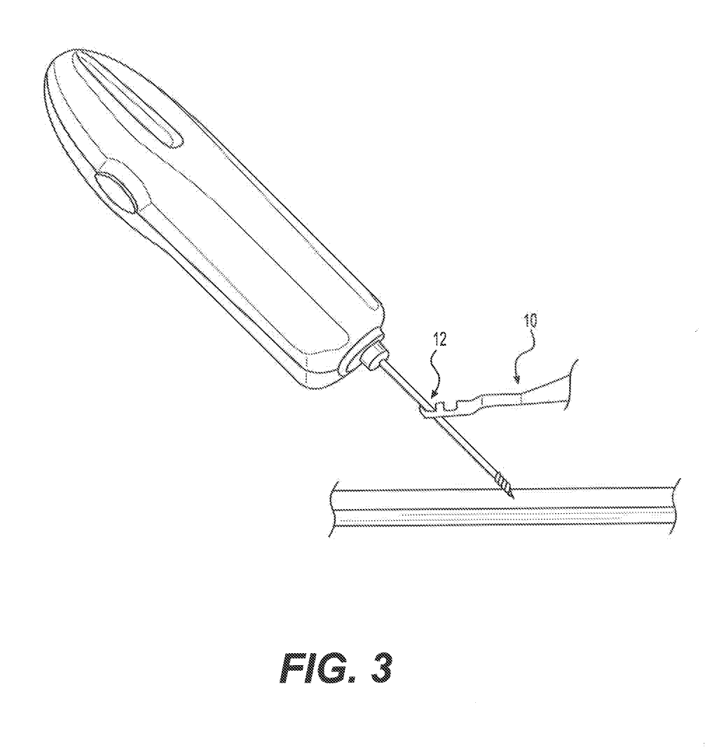

FIG. 3 illustrates loading device 10 attached to an exemplary medical conduit 12. In exemplary embodiments, attachment section 30 is designed in such a way that when force is applied to the distal end of loading device 10 in the proximal direction, loading device 10 detaches from the distal portion of medical conduit 12, but remains attached to medical conduit 12, as shown in FIG. 3. In such an embodiment, the partially detached loading device 10 is connected to medical conduit 12 in such a way that it can slide proximally along medical conduit 12 as it is inserted into the patient's body, but without interfering with the insertion or functioning of medical conduit 12.

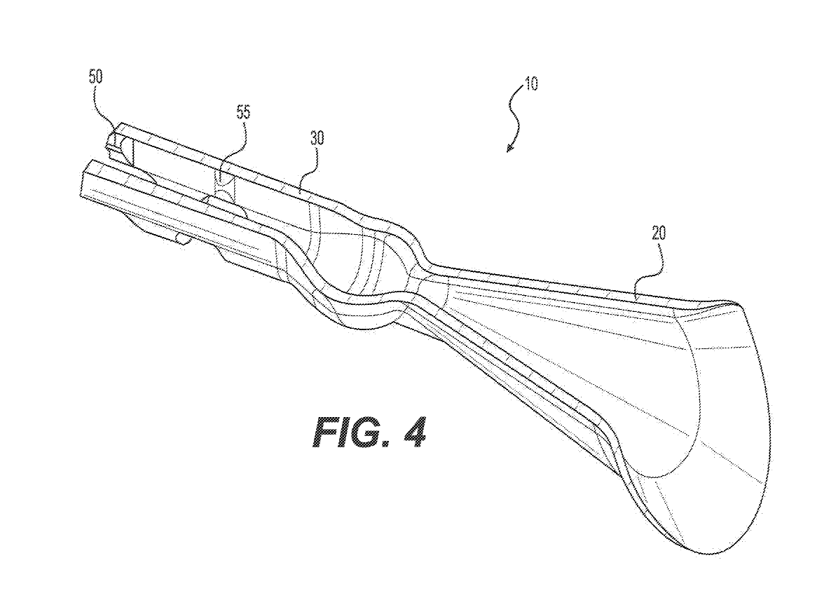

In some embodiments, attachment section 30 of loading device 10 includes one or more clips 50, as shown in FIG. 4, which may be configured to clip onto the shaft of the medical conduit onto which loading device 10 is mounted. Clips 50 may be designed to clip onto the shaft of the medical conduit in such a way that when loading device 10 moves or pivots radially outward from the medical conduit, loading device 10 remains attached to the medical conduit and can slide proximally along the medical conduit as it is inserted into the patient's body. In exemplary embodiments, clips 50 may be provided at the proximal end of attachment section 30, as shown in FIG. 4.

In some embodiments, attachment section 30 of loading device 10 includes one or more clips 55, as shown in FIG. 4, which may be configured to clip onto the shaft of the medical conduit onto which loading device 10 is mounted. Clips 55 may be designed to detach from the shaft of the medical conduit when loading device 10 moves or pivots radially outward from the medical conduit. In exemplary embodiments, clips 55 may be provided in the intermediate portion of attachment section 30, as shown in FIG. 4.

In other embodiments, attachment section 30 may include two sets of clips that may clip onto the shaft of the medical conduit. A first set of clips 55 may be provided in the middle portion of attachment section 30, and another set of clips 50 may be provided in the proximal portion of attachment section 30. In such embodiments, when force is applied to loading device 10 in the proximal direction, clips 55 may detach from the medical conduit as loading device 10 moves or pivots radially outward from the distal end of the medical conduit, while non-detachable clips 50 may remain attached to the medical conduit and slide proximally along the conduit.

In some embodiments, the portion of attachment section 30 between clips 50 and 55 may be flexible to allow loading device 10 to pivot radially outwards from the medical conduit.

A distal portion of the medical conduit may be inserted into attachment section 30. In some embodiments, the distal portion of the medical conduit may sit flush with attachment section 30. In other embodiments, the distal portion of the medical conduit may contact attachment section 30 at an angle. In one such embodiment, the distal portion of the medical conduit may contact attachment section 30 at an angle less than 60° from the long axis of loading device 10. In some embodiments, the distal portion of the medical conduit may contact attachment section 30 in only a portion of the circumference of loading device 10. In some embodiments, the portion of the circumference in which contact is made corresponds to the portion of the circumference of loading section 20 which has not been removed as distal longitudinal slot 25. In some embodiments the portion of the circumference in which contact is made corresponds to an extended region of distal tip 29 of loading section 20.

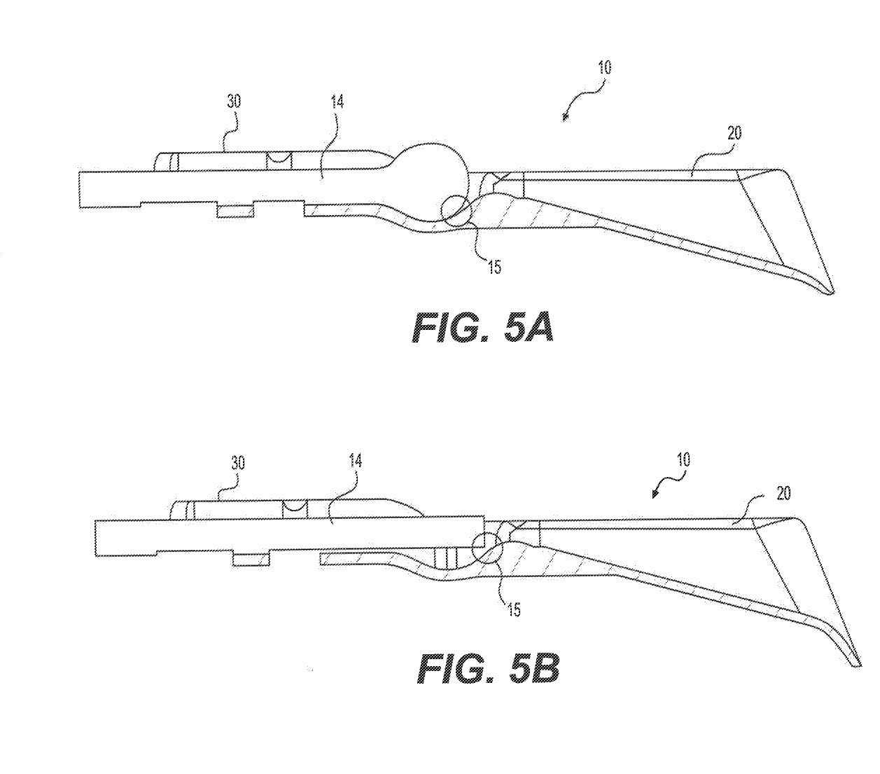

FIGS. 5A and 5B illustrate the point 15 and angle of contact between loading device 10 and an exemplary medical conduit 14. FIG. 5A shows point and angle of contact for a rounded-ended medical conduit, such as a thermal vascular closure device, and FIG. 5B shows point and angle of contact for a flat-ended medical conduit, such as a catheter. In exemplary embodiments, this point and angle of contact will induce the application of force to the distal end of loading device 10 in the proximal direction to result in an outward radial force, which causes loading device 10 to pivot or rotate outward, which then causes loading device 10 to detach partially or completely from the distal end of medical conduit 14.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments.

Moreover, while illustrative embodiments have been described herein, the scope of any and all embodiments include equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

1. A device for loading a medical conduit onto a guidewire, comprising:
an incomplete tubular attachment portion configured to fit over an end portion of the medical conduit, the tubular attachment portion having a longitudinal section removed;
an incomplete conical loading portion extending from the tubular attachment portion, the conical loading portion having a longitudinal section removed;
wherein the conical loading portion is configured to direct the guidewire into a lumen of the medical conduit; and
further wherein the device is configured to be detachable from the medical conduit after the guidewire is directed into the medical conduit.

2. The device of claim 1, wherein the device is further configured to pivot away from the medical conduit when force is applied to a distal end of the conical loading portion.

3. The device of claim 1, wherein the device is further configured to be completely detachable from the medical conduit.

4. The device of claim 1, wherein the device is further configured to be partially detachable from the medical conduit.

5. The device of claim 1, wherein the removed longitudinal section of the tubular attachment portion corresponds to approximately half of a tube.

6. The device of claim 1, wherein the removed longitudinal section of the tubular attachment portion corresponds to approximately one-third of a tube.

7. The device of claim 1, wherein the removed longitudinal section of the conical loading portion corresponds to approximately half of a cone.

8. The device of claim 1, wherein the removed longitudinal section of he conical loading portion corresponds to approximately one-third of a cone.

9. The device of claim 1, wherein the tubular attachment portion comprises one or more detachable clips that are configured to detach from the medical conduit when force is applied to a distal end of the conical loading portion.

10. The device of claim 9, wherein the one or more detachable clips are provided at a midpoint of the tubular attachment portion.

11. The device of claim 1, further comprising one or more non-detachable clips at a proximal end of the tubular attachment portion.

12. The device of claim 11, wherein the one or more non-detachable clips are configured to keep the proximal end of the tubular attachment portion connected to the medical conduit when the rest of the device detaches and pivots away from the medical conduit.

13. The device of claim 1, further comprising one or more non-detachable clips at a proximal end of the tubular attachment portion and one or more detachable clips at a midpoint of the tubular attachment portion.

14. The device of claim 1, wherein a distal tip of the conical loading portion extends further distally at its midpoint than at its sides.

15. The device of claim 1, wherein a continuous lumen extends through the tubular attachment portion and the conical loading portion.

16. The device of claim 1, wherein the incomplete tubular portion constitutes a proximal section of the device and the incomplete conical portion constitutes a distal section of the device when the device is fitted to the end portion of the medical conduit.
